(19) United States
(12) Patent Application Publication
(10) Pub. No.: US 2008/0009953 A1
(43) Pub. Date: Jan. 10, 2008

(54) Flanged prosthetic acetabular cup

(75) Inventors: Robin Sydney Mackwood Ling, Somerset (GB); Jonathan Howell, Cullompton (GB); Andrew John Timperley, St. Leonard's Exeter (GB); Matthew Hubble, Exeter (GB); Graham Allan Gie, Crediton (GB); Philippe Lavieille, Caen (FR); Pascal Collet, Saint-Martin-De-Fontenay (FR); Alain Richard, Epron (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Benoist Girard SAS, Herouville-saint-clair Cedex (FR)

(21) Appl. No.: 11/786,756

(22) Filed: Apr. 12, 2007

(30) Foreign Application Priority Data
May 3, 2006 (GB) 0608756.3

(51) Int. Cl. A61F 2/32

(52) U.S. Cl. 623/22.21

(57) ABSTRACT

A kit and a method for implanting a prosthetic acetabular cup in an acetabular has a prosthetic acetabular cup having an open end for receiving a head of a prosthetic femoral component. The cup has a flange extending outwardly from a surface thereof adjacent the open end. The flange is angled toward the acetabulum. The acetabulum is prepared to receive the prosthetic acetabular cup, the preparation includes forming a circumferential rim shaped seat in the bone around the acetabulum. The cup flange has a maximum diameter less than the outer diameter of the circumferential rim.

### BACKGROUND OF THE INVENTION

This invention relates to a prosthetic acetabular cup which has a peripheral outwardly projecting flange. Flanged cups are known, the flange being provided to prevent the escape of cement around the upper periphery of the cup when it is fitted. The flange also allows for the cement to be pressurized. The typical diametric width of such flanges which project from the outer surface of the cup is usually about 18 to 25 mm. Such cups are shown in U.S. Pat. Nos. 4,324,006, 4,327,449 and 6,019,766. The disclosure of U.S. Pat. No. 6,019,766 is incorporated herein by reference.

It has been found that if an accurate acetabular rim cutter is used on the bone then the outer edges of an acetabulum can be machined to provide an annular seating surface or cavity against which a flanged acetabular cup can seat. The use of such an accurate rim cutter can allow for the use of a flange of much smaller overall diameter than those which have been used previously.

A rim cutter of the kind referred to above is shown in U.S. Patent Publication No. 20060079906, the disclosure of which is incorporated herein by reference.

### SUMMARY OF THE INVENTION

According to the present invention a prosthetic acetabular cup has a peripheral outwardly projecting flange, the diametrical width of which is between 4 and 6 mm. Such a flange is of a width considerably smaller than those which have been used previously. In a preferred embodiment the diametric width is 5 mm. Preferably the flange extends away from the mouth of the cup in a proximal direction at an angle of approximately 30°. The outer circumference of the flange can be spaced away from the outer rim of the mouth of the cup by approximately 1.8 mm. Preferably the flange is circular.

The outer surface of the cup can be provided with indentations to assist in adhesion with the cement and the cup can incorporate an x-ray marker. The flange can have a thickness of approximately 1.0 mm. The cup can be made from any suitable material, for example a synthetic plastics material, composite material, metal or a combination thereof.

The invention also includes a kit of parts comprising a prosthetic acetabular cup as set forth above and a trial cup, the dimensions of the flange of which are similar to the prosthetic acetabular cup and which include a projection or projections on its outer surface to represent a cement level in the acetabulum with which the cup is to be used. In a preferred construction the projection is provided by an annular abutment around an outer circumference of the cup in a plane perpendicular to a polar axis of the cup.

As used herein when referring to bones or other parts of the body, the term “proximal” means closer to the heart and the term “distal” means more distant from the heart. The term “inferior” means toward the feet and the term “superior” means toward the head. The term “anterior” means toward the front part or the face and the term “posterior” means toward the back of the body. The term “medial” means toward the midline of the body and the term “lateral” means away from the midline of the body.

A method and a kit for implanting a prosthetic acetabular cup in an acetabulum includes providing a prosthetic acetabular cup having an open end for receiving a head of a prosthetic femoral component. The cup has a flange extending outwardly around the circumference thereof from a surface thereof adjacent the open end. The flange is angled toward the acetabulum. The acetabulum is prepared to receive the prosthetic acetabular cup with the preparation including forming a circumferential rim shaped seat in the bone surrounding the acetabulum. The cup flange has a maximum outer diameter greater than the opening in the acetabulum and less than the diameter of the circumferential rim formed in the bone. The angle of the flange extending from the open end of the acetabular cup extends at an angle of less than 60° and preferably 30° towards the rim formed around the acetabular opening. Preferably the outer diameter of the cup flange is about 1 mm less than the diameter of the circumferential rim. Preferably the flange has an inner circumference adjacent the outer cup surface which is 1.8 to 2 mm from the mouth of the acetabulum so that the outer circumference of the cup may be spaced a sufficient distance to form a bone cement mantle. In addition, the flange is approximately 1 mm thick and is made of ultrahigh molecular weight polyethylene.

A kit may be provided which includes a trial acetabular cup also having an open end for receiving the head of a prosthetic femoral component and having a flange extending outwardly from a part-spherical outer surface thereof adjacent the open end. Again the flange is angled towards the acetabulum. A series of different sized trial inserts can be utilized each having different dimensions for engaging different sized acetabulums.

### BRIEF DESCRIPTION OF THE DRAWINGS

The invention can be performed in various ways but one embodiment will now be described by way of example and with references to the accompanying drawings in which:

FIG. 1 is a plan view of the cup of the present invention;

FIG. 2 is a cross-sectional elevation of a prosthetic acetabular cup, according to the present invention, on the line II-II of FIG. 1:

FIG. 3 is a plan view from beneath of the cup shown in FIGS. 1 and 2;

FIG. 4 shows the metallic X-ray marker which is incorporated in the cup;

FIG. 5 is a side view of a trial cup for use with the cup shown in FIGS. 1 to 4; and

FIG. 6 shows how the cup, according to the invention, is located in an acetabulum.

### DETAILED DESCRIPTION

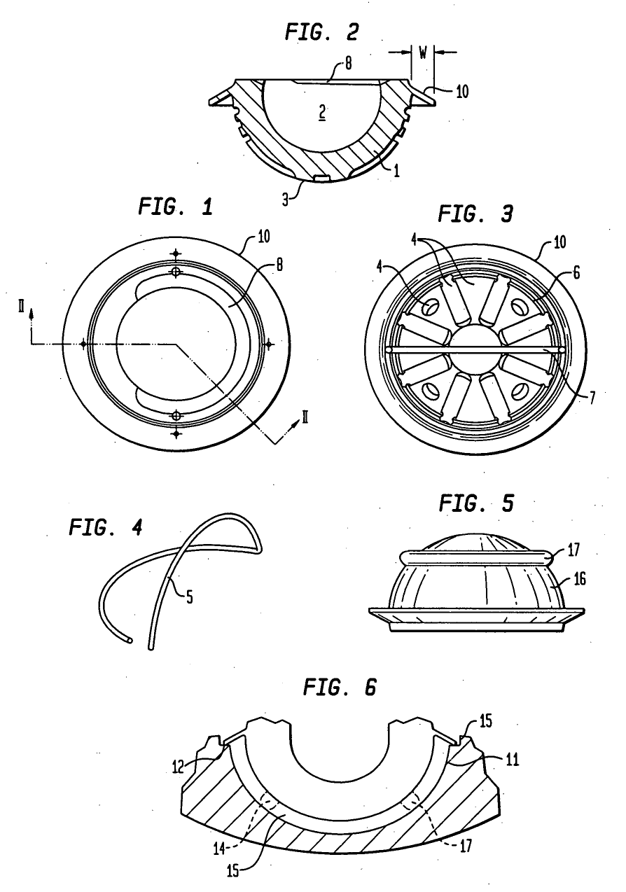

As shown in FIGS. 1 to 4 the prosthetic acetabular cup 1 according to the invention has a part-spherical inner bearing surface 2 and an outer surface 3 which is provided with a series of indentations 4 and projections to assist in anchoring it in the cement. The cup is provided with an X-ray marker 5, as shown in FIG. 4, and which can be located in appropriate grooves 6 and 7 on the outer surface.

The mouth of the bearing surface 2 has a chamfer 8 over part of its circumference.

Cup 1 is provided with a peripheral outwardly projecting flange 10, the diametric width, which is indicated by reference letter W, is between 4 and 6 mm. In a preferred construction the width is 5 mm. The flange extends away from the mount of the cup in a proximal direction at an angle of less than 60° and preferably approximately 30° and the inner circumference of the flange is spaced away from the outer rim of the mouth of the acetabular cup by approximately 1.8 mm to 2.0 mm.

The flange is circular, as will be seen from FIGS. 2 and 3, and it has a thickness of approximately 1.0 mm.

FIG. 6 shows how the mouth of the acetabulum 11 into which cup 1 is to be fitted is trimmed by machining an annular seating surface 12, as shown in FIG. 6. Annular seating surface 12 is machined by an acetabular rim cutter, for example of the type shown in U.S. Patent Publication 20060079906. Flange 10 of acetabular cup 1 can now be seated on annular seating surface 12.

A gap 13 is left between the outer surface of the cup and the inner surface of the acetabulum to accept the cement. If required, spacers in the form of projections 14 could also be provided on the cup to maintain gap 13.

The use of the rim cutter, as described above, enables an annular flange on the cup to be of much reduced diameter compared with those which have been used previously and ensures that the edge of the flange can also be located on annular seating surface 12 which, as shown in FIG. 6, can be cut into the bone to provide a cavity with an annular location rim 15. The diameter of flange 10 can be about 1 mm less than the diameter of location rim 15 of the cavity. The use of the small diameter flange and the cutter enables such a cut to be made which would not be possible using a large diameter flange of known type.

The invention also includes a kit of parts which comprises the cup as set forth above and a trial cup. The trial cup has dimensions and flanges similar to that in the cup described above and also include a projection or projections 14 on its outer surface to represent a cement level 13 in acetabulum 11 with which cup 1 is to be used.

As will be seen from FIG. 5 the projection or projections can be provided by an annular abutment 17 which is also shown in broken lines in FIG. 6. The trial cup 16 is used by the surgeon to confirm the amount of cut required on the rim of the acetabulum during fitting of the cup according to the invention.

The use of the small flange guarantees a homogeneous cement mantle and it is possible to perform a reduction trial because the rim cavity ensures good stability of the cup.

Thanks to the precisely shaped flange surgeons do not need any cutting step and the diameter of the flange can correspond precisely with the rim cavity (see FIG. 6). This perfect fit between the flange and the cavity leads to an optimal pressurization of the cement.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method for implanting a prosthetic acetabular cup in an acetabulum comprising:
providing a prosthetic acetabular cup having an open end for receiving a head of a prosthetic femoral component, the cup having a flange extending outwardly from an outer circumferential surface thereof adjacent the open end the flange angled toward the acetabulum, the angle being constant around the circumference;
preparing the acetabulum to receive the prosthetic acetabular cup, the preparation including forming a circumferential rim shaped seat in the bone surrounding the acetabulum, the cup flange having an outer diameter greater than a maximum diameter of an opening at the acetabulum and less than an outer diameter of the circumferential rim; and
implanting the acetabular cup with the cup flange contacting the rims.

2. The method as set forth in claim 1 wherein the angle of the flange extending from the open end toward the acetabulum is less than 60° when measured from a planar surface across the open end of the cup.

3. The method as set forth in claim 1 wherein the outer diameter of the cup flange is about 1 mm less than the outer diameter of the circumferential rim.

4. The method as set forth in claim 1 wherein the flange has an inner circumference spaced inwardly about 1.8 to 2.0 mm from the maximum outer diameter of the opening of the acetabulam.

5. The method as set forth in claim 1 wherein the flange is about 1 mm thick.

6. The method as set forth in claim 1 further comprising using a trial acetabular cup similar in design of the prosthetic acetabular cup to size the acetabular.

7. The method for implanting an acetabular cup as set forth in claim 1 wherein the cup flange has a width between its inner and outer diameter of 4 to 6 mm.

8. A kit for use in implanting a prosthetic acetabular cup comprising:
a plurality of trial acetabular cups each having an open end for receiving a head of a prosthetic component the cups having a flange extending outwardly from an outer surface thereof adjacent the open end, the flanges angled toward the acetabulum at a constant angle around a circumference of the open end; and
a plurality of prosthetic acetabular cups for implantation into the acetabulum each cup having an open end for receiving a head of a prosthetic femoral component, cup having a flange extending from an outer surface thereof adjacent the open end which flange is angled toward the acetabulum, the sizes of the prosthetic acetabular cups corresponding to the sizes of the trial acetabular cups.

9. The kit as set forth in claim 8 wherein the flange has an inner circumference spaced inwardly about 1.8 to 2.0 mm from the maximum opening of the acetabulum.

10. The kit as set forth in claim 8 wherein the angle of the flange extending from the open end of the trial and the prosthetic acetabular cups extend at an angle of less than 60° when measured from a planar surface across the open end of the cup.

11. The kit as set forth in claim 8 wherein the trial and prosthetic acetabular cup has a flange with a width of 4 to 6 mm when measured from an outer diameter of the flange to an outer surface of the cup.

12. The kit as set forth in claim 9 further comprising an acetabular rim cutter for forming an annular seat around the opening in the acetabulum.
